(19) United States
(12) Patent Application Publication
(10) Pub. No.: US 2005/0084525 A1
(43) Pub. Date: Apr. 21, 2005

(54) Method for blinding a medication

(76) Inventors: Adeyinka A. Abinusawa, North Wales, PA (US); Abizer H. Bookwala, Harleysville, PA (US); Ashok V. Katdare, Highland Park, IL (US); Patrick E. McLaughlin, Doylestown, PA (US)

Correspondence Address: MERCK AND CO., INC, P O BOX 2000, RAHWAY, NJ, 07065-0907, US

(21) Appl. No.: 10/933,092

(22) Filed: Sep. 1, 2004

Related U.S. Application Data
(60) Provisional application No. 60/512,726, filed on Oct. 20, 2003.

(51) Int. Cl. A61K 9/48; A61K 9/64; A61K 9/20; A61J 1/00

(52) U.S. Cl. 424/456; 424/464; 427/2.14

(57) ABSTRACT

An object of the present invention is to provide an improved method for blinding a dosage form such that investigators analyzing such dosage form and subjects ingesting such dosage form cannot determine the identity of the active medication from the placebo they are administering or ingesting. The improved method is termed “overprinting”. In an embodiment, the method for blinding a dosage form comprises printing one or more layers of ink to said dosage form such that the original markings on said dosage form are masked.

### BACKGROUND OF THE INVENTION

The present invention relates to a method for making a blinded oral dosage form, used for disguising the identity of medications used in clinical trials.

Over the years, various methods and devices have been used to compare one medication with another to determine the relative safety and/or efficacy of the medication. It is a common practice to disguise the medication in order to prevent any prejudice from investigators administering the drugs and subjects ingesting the drugs. Disguising medications from investigators administering or subjects ingesting the medication is commonly referred to as “blinding”.

Blinding of oral solid dosage forms with distinguishing features poses a challenge in clinical trials and has been accomplished in the past by methods including, overencapsulation, deprinting, overcoating and a grinding method followed by recompression or encapsulation, all of which have significant drawbacks.

Overencapsulation is the process of placing a “dosage form” or “medication” (tablet, hard and soft gelatin capsules and the like) into an appropriate size capsule with or without an inert material (backfill) including but not limited to cellulose, lactose or starch or a combination thereof, to fill up the excess space formed between the inserted dosage form and the walls of the overencapsulating capsule. Problems associated with overencapsulation are many. Drug bioavailability and/or dissolution may be altered due to the presence of gelatin and/or backfill material. Overencapsulation involves multiple unit operations such as weighing, encapsulation, weight sorting, inspection and capsule polishing. Additionally, analytical testing and stability studies (for example, dissolution and degradation tests) may be needed to evaluate the impact of overencapsulation on drug stability and bioavailability. Overencapsulation significantly increases the size and weight of the original dosage form and can directly influence clinical study outcomes e.g. patient compliance may decrease with larger dosage forms, thus influencing the outcome of a clinical study. Finally, the increase in dosage size can be a burden on downstream operations such as packaging and warehousing.

Deprinting is the process of removing ink from a dosage form or medication (tablet, hard and soft gelatin capsules and the like). This can be accomplished by using a suitable aqueous or volatile solvent to remove the ink markings. The process of deprinting is slow and labor intensive and does not guarantee complete removal of the ink. Deprinting can also remove the film-coat on the dosage form and potentially expose the granulation. Since deprinting may remove all or a portion of the coating polymer, the process cannot be used for dosage forms with functional coats. Also, the deprinted portion may appear significantly different from the rest of the dosage form, making it difficult to manufacture a matching placebo. Deprinting may involve the use of volatile organic solvents which require special handling and disposition thereby increasing costs. Not all markings can be removed by deprinting especially those formed by inks which penetrate or “bite” or adhere strongly to the dosage form.

Thus, there remains a long felt need in the art for an improved method for blinding dosage forms. A new method of blinding would have many advantages including; preservation of the bioavailability of the dosage form after blinding; preservation of the stability of the dosage form after blinding; the blinding method would be quicker and more efficient; the blinding method would be independent of dosage form weight and size; and there should be no increase in the size and weight of the dosage form after blinding thus patient compliance would be unaffected.

### BRIEF DESCRIPTION OF THE FIGURE

FIG. 1: Process Flow

The figure demonstrates an embodiment of the procedure utilized in the instant invention encompassing product and equipment preparation and ultimate overprinting.

### SUMMARY OF THE INVENTION

An object of the present invention is to provide an improved method for blinding a solid oral dosage form such that investigators analyzing such dosage form and subjects ingesting such dosage form cannot distinguish the identity of the placebo from the active medication they are administering or ingesting. The improved method is termed “overprinting”.

In an embodiment of the instant invention is provided a method for blinding a dosage form comprising printing one or more layers of ink to said dosage form such that the original markings on said dosage form are masked.

In another embodiment, the dosage form comprises a formulated medication or a placebo.

In still another embodiment, the dosage form comprises a formulated medication or a placebo used to conduct clinical trials.

In yet another embodiment, the dosage form is selected from: a tablet; a soft gelatin capsule; and a hard gelatin capsule.

In a further embodiment, the dosage form is a hard gelatin capsule.

In another embodiment, said printing is achieved by a process selected from: spin printing and linear printing.

In another embodiment, said printing is achieved by spin printing.

In another embodiment, the ink is edible pharmaceutical printing ink.

In another embodiment of the instant invention is provided a method for blinding a dosage form via an overprinting process comprising the steps of:
a) loading said dosage form into a printing machine;
b) adjusting the print speed on said printing machine;
c) adjusting the print location on said printing machine;
d) printing; and
e) repeating said overprinting process as necessary to achieve complete masking.

In another embodiment, the printing machine is selected from: a spin printing machine and a linear printing machine.

In another embodiment, the printing machine is a spin printing machine.

In another embodiment, the dosage form comprises a formulated medication or a placebo.

In another embodiment, the dosage form comprises a formulated medication or a placebo used to conduct clinical trials.

In another embodiment, the dosage form is selected from: a tablet; a soft gelatin capsule; and a hard gelatin capsule.

In another embodiment, the dosage form is a hard gelatin capsule.

In another embodiment, the print speed ranges from about 2 to about 20 rpm.

In another embodiment, the print speed ranges from about 5 to about 12 rpm.

In another embodiment, the print location is adjusted by modifying the print registration and print height on said printing machine.

In another embodiment of the instant invention is provided a method for blinding a dosage form via an overprinting process comprising the steps of:
a) preparing said dosage form to be loaded into a printing machine;
b) staging said dosage form into said printing machine;
c) printing; and
d) repeating said printing as necessary to achieve complete masking.

In another embodiment, the dosage form comprises a formulated medication or a placebo used to conduct clinical trials.

In another embodiment, said placebo is filled with inert excipients.

In another embodiment, said formulated medication is a comparator which is removed from a market container.

In another embodiment, said staging occurs in said printing machine hopper.

In another embodiment, the printing machine is selected from: a spin printing machine and a linear printing machine.

In another embodiment, the printing machine is a spin printing machine.

In another embodiment, the dosage form is selected from: a tablet; a soft gelatin capsule; and a hard gelatin capsule.

### DETAILED DESCRIPTION OF THE INVENTION

The following description of various embodiments of the method for blinding a medication are merely illustrative of the present invention and they should not be considered as limiting the scope of the invention in any way, as these illustrations and other equivalents thereof will become more apparent to those versed in the art in the light of the present disclosure, and the accompanying claims.

The method for blinding a dosage form comprises a technique whereby one or more layers of ink are deposited on specific portions of solid dosage form (e.g. tablets and hard and soft gelatin capsules and the like) for the purpose of blinding the original marking(s) on the dosage form. The utility of such a blinded dosage form is in the field of clinical trials where regulations permit and encourage the use of blinded products (dosage forms) so as to eliminate patient or investigator bias in the final outcomes.

Accordingly, the method for blinding a dosage form comprises printing one or more layers of ink to said dosage form such that the original markings on said dosage form are masked.

A “dosage form” means any solid dosage of medicine or drug packaged for consumption in various well known forms, such as tablets (including coated tablets), caplets, capsules (including coated capsules), soft gelatin capsules, hard gelatin capsules, sachets and others.

A “formulated medication” means a mixture or prescribed recipe for packaging a pharmaceutical. A “placebo” means any dummy medication or treatment. Although placebos originally were medicinal preparations having no specific pharmacological activity against a targeted condition, the concept has been extended to include treatments or procedures, especially those administered to control groups in clinical trials in order to provide baseline measurements for the experimental protocol. In one aspect of the invention, a dosage form comprises a solid dosage formulated medication or a placebo. In another aspect of the invention, a dosage form comprises a formulated medication or a placebo used to conduct clinical trials. In yet another aspect of the invention, the dosage form is selected from: a tablet; a soft gelatin capsule; and a hard gelatin capsule. In still another aspect of the invention, the dosage form is a hard gelatin capsule.

“Printing” means a process for the application of a suitable ink formulation to the dosage form. The printing process can be accomplished by any means well known in the art including linear and spin printing.

Spin printing is carried out by rotating the dosage form as ink is printed on the surface. Numerous methods and machines have been developed to spin print ink onto dosage forms. In general, there are three types of methods and corresponding apparatuses for spin printing. The first apparatus and method spin prints using a flat type carrier apparatus, e.g., conveyor belt. Examples of the conveyor belt type of spin printing apparatus are disclosed in U.S. Pat. Nos. 3,868,900; 3,871,295; 3,931,884; 4,069,753; 4,104,966; 4,167,226; 4,254,704; 4,266,477; 4,335,810; 4,372,437; 4,413,556; 4,479,573 and 4,632,028. The second apparatus and method spin prints onto the dosage form using rotating drums or cylinders. Examples of the rotating drum type of spin printing apparatus are disclosed in U.S. Pat. Nos. 3,889,591; 4,266,478; 4,369,702; 4,377,971 and 4,394,933. The third type of spin printing apparatus and method uses a conveyor and a printing roller as disclosed in U.S. Pat. No. 6,286,421 which is hereby incorporated by reference.

The spin printing apparatus and method which uses a conveyor and a printing roller, as disclosed in U.S. Pat. No. 6,286,421 is the preferred apparatus and method. Moreover, the printing step may be repeated as necessary to ensure complete masking of the original markings on the dosage forms.

In another aspect of the invention, printing involves the application of ink via a printing process well known in the art. In yet another aspect of the invention, printing involves the application of ink via spin printing or linear printing. In still another aspect of the invention, printing involves the application of ink via spin printing.

“Ink” means any edible pharmaceutical printing ink well known in the art, including ink made with dry shellac, ethyl alcohol, plasticizers and/or detackifiers, pigments, or dyes and solvents and others. Moreover, the printing ink can be of any suitable color appropriate for masking. Examples of edible ink include Opacode Black # S1R-8100HV, Opacode Black # S-1-17780, Opacode Blue # S-1-4176, Opacode Black # S-1-17768 and Opadry Black S-1-17768. Examples of edible ink are further disclosed in U.S. Pat. Nos. 3,258,347 and 4,543,370. Therefore, in another aspect of the invention, the ink is edible pharmaceutical printing ink well known in the art.

A dosage form is “masked” when the applied ink covers the entire area of a dosage form wherein the “original markings” (i.e., logos, writings, trademarks and tradenames) are blocked out such that an investigator and/or a subject cannot determine whether the dosage form is the actual dosage form containing a drug or placebo. Typically, a masked dosage form has up to a 360° “band” of ink which completely covers the original markings. As needed, a masked dosage form may have more than one band. Moreover, the band may be of any width necessary to cover the original markings.

The following example is not limiting but rather illustrative of the method of this invention.

### EXAMPLE

Materials and Equipment

Size #1 white opaque hard gelatin capsules with blue ribbon bands
Size #2 white opaque hard gelatin capsules
Size #2 light blue opaque hard gelatin capsules
Starch NF
Comparator capsule #1
Comparator capsule #2
Comparator capsule #3
Comparator capsule #4
H&K Encapsulator
Ackley VIP II printing machine (linear and spin printing).
   Method

Prior to printing trials, approximately 100,000 size #1 white opaque placebo hard gelatin capsules and 50,000 size #2 white opaque placebo hard gelatin capsules were filled with starch NF on an H&K 400 Encapsulator. Additionally approximately 10 Kg of size #2 blue opaque placebo capsules were utilized. These placebo capsules along with four different comparator capsules (selected to represent examples that may be encountered during blinding of clinical trial material) were used in evaluating machine speeds and ink formulations during the feasibility trials.

The size #1 white opaque capsules contain a design which includes a blue band surrounding the cap and body with the numbers “4042” and “912” printed in blue ink on the cap and body respectively. Both size #2 capsules contain no markings.

During the trials, the placebo capsules described above and the comparator capsules (#1) were overprinted with three different ink formulations; Opacode Black S-R-8100 HV, Opacode Black S-1-17768 and Opacode Blue S-1-4176. Further during the trials Opacode Black S-R-8100 HV and Opacode Black S-1-17780 were used on three additional comparator capsules (#2, #3 and #4).

A listing of process variables identified during the trials is presented below:

1. Ink formulations:

Results showed that Opacode Black S1R-8100 HV and Opacode Black S-17780 provided optimal drying times. Comparator capsules may need to be printed twice to achieve satisfactory coverage “masking”.

The components of all inks are presented in Table 1. The ink formulations contain dyes acceptable to E.U. and U.S. regulatory formulations. Denatured alcohol (SDA-3A) a solvent used in the inks differs from denatured alcohol in most European countries. This is because of differences in the regulatory classification between the two regions. Given the role and necessity of a robust ink formulation to achieve occlusion, the printing ink was designated a critical process variable.

TABLE 1
Components of Opacode Inks

| | Opacode | Opacode | Opacode | Opacode |
|---|---|---|---|---|
| | Black | Black | Blue | Black |
| Ingredients | # S1R-8100HV | # S-1-17780 | S-1-4176 | S-1-17768 |
| Shellac Glaze | ✓ | ✓ | ✓ | ✓ |
| (modified) | | | | |
| in SD-45 | | | | |
| FD&C Blue #1 | — | — | ✓ | — |
| Aluminum lake | | | | |
| -ADI- | | | | |
| 0-12.5 mg | | | | |
| FD&C Blue #2 | | | | ✓ |
| Aluminum lake | | | | |
| -ADI- | | | | |
| 0-5 mg | | | | |
| Synthetic black | ✓ | ✓ | — | ✓ |
| iron oxide | | | | |
| -ADI- 0-0.5 mg | | | | |
| Propylene Glycol | — | ✓ | ✓ | ✓ |
| SDA 3A Alcohol | ✓ | ✓ | ✓ | ✓ |
| N-Butyl Alcohol | — | ✓ | ✓ | ✓ |
| Isopropyl Alcohol | — | ✓ | — | ✓ |
| Ammonium | — | ✓ | — | ✓ |
| Hydroxide | | | | |
| Purified Water | ✓ | — | — | — |
| 2-Ethoxyethanol | ✓ | — | — | — |
| Lecithin | ✓ | — | — | — |
| Simethicone | ✓ | — | — | — |

Dyes have an Acceptable Daily Intake (ADI), an estimate of the amount of a substance in food or drinking water, expressed on a body-weight basis. The ADI is indicated in units of mg/kg of body weight.

2. Printing Speeds:

Machine speeds between 5 rpm (22,500 capsules/hour) and 12 rpm (54,000 capsules/hour) were found adequate for the process using Opacode Black SIR-8100 HV on all placebo and comparator product capsules.

At slower speeds (between 3-4 rpm) the ink begins to dry before depositing on capsules. At higher speeds (>15 rpm) the capsule dwell time within the print zone is too short for efficient ink transfer. Also the drying time (time between ink deposition and capsule ejection into collection chute) may not be adequate at high speeds. Based on these observations the printing speed was also deemed a critical process variable. It should be noted that an ink formulation could possibly be optimized to allow faster processing but the marginal utility or value of such a reformulation may not be significant. The likelihood of capsules flying out of the carrier link at higher speeds also reduces any benefits.

Given the fact that some capsules may need to be printed twice, the effective operating speed range then becomes 11,250-27,000 capsules per hour. These speeds are still significantly faster compared to deprinting or overencapsulation.

Other Process Variables:

The following process parameters were also evaluated during the printing trials.

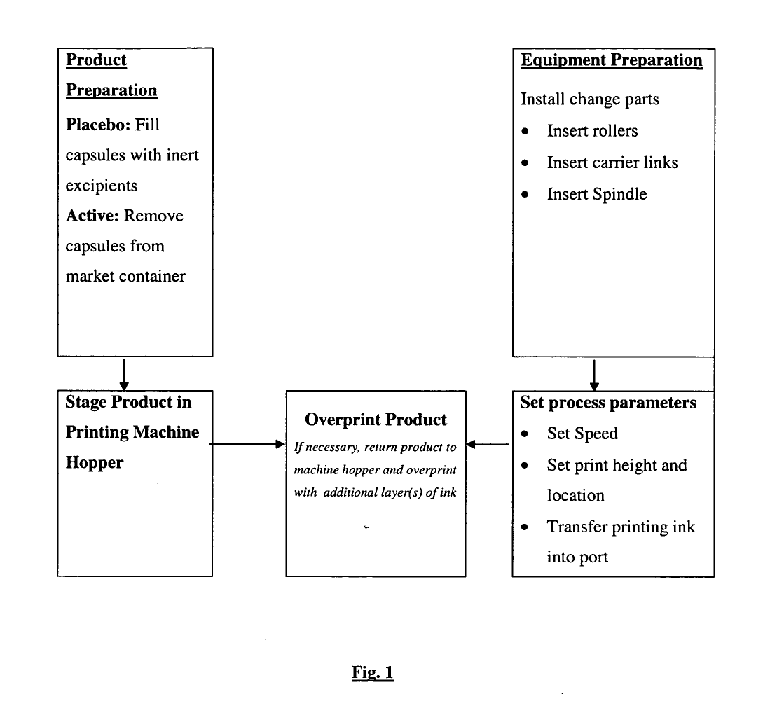

A process flow chart is presented in FIG. 1.

3.0. Print Location:

The location of the print on the comparator capsule is crucial to achieve successful blinding. Complete ink transfer depends on ability of the offset rollers to come in contact with a comparator at a precise location where coverage is desired. This positioning of the offset rollers depends on two machine parameters, discussed below.

3.1. Print Registration:

This is an electronically controlled variable in the VIP II that controls location (centering) of the over print logo on the dosage form. It consists of a fine and coarse adjustment mechanism and is measured in inches.

3.2. Print Height:

This is a manual adjustment that moves the offset rollers towards or away from the capsule during printing. This controls how much ink is deposited and also determines the pressure being applied on the dosage form within the print zone.

Capsule Weight Gain

Change in capsule weight as a function of overprinting was investigated. Twelve unprinted capsules each of comparator capsules (#2, #3 and #4) were numbered, weighed individually and then over printed. The comparator capsules (#2) were printed with Opacode Black S-IR-8100 HV while comparator capsules (#3 and #4) were printed with Opacode Black S-1-17768. The capsules were allowed to air dry for approximately 96 hours and then re-weighed on the same balance. Results presented in Tables 2-4 show that the change in capsule weight after overprinting is insignificant and the amount (mg) of ink material deposited may be considered undetectable.

TABLE 2
Weight Gain for Comparator Capsules (#2) Printed
with Opacode Black S-1R-8100HV

| | Individual capsule | | |
|---|---|---|---|
| | weights (mg) | | Change in capsule |
| Capsule # | Before printing | After overprinting | weight (mg) |
| 1 | 323.6 | 323.7 | 0.1 |
| 2 | 323.6 | 322.8 | −0.8 |
| 3 | 319.0 | 319.0 | 0.0 |
| 4 | 325.3 | 325.2 | −0.1 |
| 5 | 322.1 | 321.4 | −0.7 |
| 6 | 309.4 | 309.6 | 0.2 |
| 7 | 318.8 | 318.8 | 0.0 |
| 8 | 316.3 | 315.4 | 0.9 |
| 9 | 314.3 | 314.3 | 0.0 |
| 10 | 320.7 | 320.6 | −0.1 |
| 11 | 320.1 | 320.8 | 0.7 |
| 12 | 314.4 | 315.1 | 0.7 |
| Average | 319.0 | 318.9 | −0.09 |
| % RSD | 1.46 | 1.42 | |

TABLE 3
Comparator Capsules (#3) Printed with Opacode Black S-1-17768

| | Individual capsule | | |
|---|---|---|---|
| | weights (mg) | | Change in capsule |
| Capsule # | Before printing | After overprinting | weight (mg) |
| 1 | 318.7 | 318.6 | −0.1 |
| 2 | 312.2 | 312.2 | 0.0 |
| 3 | 310.5 | 310.7 | 0.2 |
| 4 | 311.0 | 311.3 | 0.3 |
| 5 | 313.2 | 314.0 | 0.8 |
| 6 | 311.3 | 311.5 | 0.2 |
| 7 | 321.5 | 321.6 | 0.1 |
| 8 | 325.0 | 325.4 | 0.4 |
| 9 | 315.9 | 316.5 | 0.6 |
| 10 | 317.4 | 317.9 | 0.5 |
| 11 | 309.6 | 310.0 | 0.4 |
| 12 | 315.3 | 315.9 | 0.6 |
| Average | 315.1 | 315.5 | 0.30 |
| % RSD | 1.52 | 1.52 | |

TABLE 4
Comparator Capsules (#4) Printed with Opacode Black S-1-17768

| | Individual capsule | | |
|---|---|---|---|
| | weights (mg) | | Change in capsule |
| Capsule # | Before printing | After overprinting | weight (mg) |
| 1 | 315.0 | 315.1 | 0.1 |
| 2 | 319.5 | 318.9 | −0.6 |
| 3 | 303.2 | 303.2 | 0.0 |
| 4 | 318.3 | 318.4 | 0.1 |
| 5 | 310.9 | 310.9 | 0.0 |
| 6 | 319.6 | 320.2 | 0.6 |
| 7 | 315.7 | 315.6 | −0.1 |
| 8 | 315.8 | 315.6 | −0.2 |
| 9 | 321.4 | 320.8 | −0.6 |
| 10 | 313.7 | 313.6 | −0.1 |
| 11 | 320.7 | 320.4 | −0.3 |
| 12 | 317.6 | 317.2 | −0.4 |
| Average | 316.0 | 315.8 | −0.13 |
| % RSD | 1.60 | 1.57 | |

Stability

The components of the Opacode ink formulations (Table 1) used for overprinting are common to Pharmaceutical grade inks and may theoretically not be considered as sources of capsule or product degradation.

### CONCLUSION

Overprinting of comparator hard gelatin capsules was evaluated and successfully developed as a new process for manufacturing blinded material for clinical trials. This process offers unique advantages compared to the currently available methods. Critical process variables identified were ink formulation and machine speeds.

From the foregoing description, one of ordinary skill in the art can easily ascertain the essential characteristics of the instant invention, and without departing from the spirit and scope thereof, can make various changes and/or modifications of the invention to adapt it to various usages and conditions. As such, these changes and/or modifications are properly, equitably and intended to be, within the full range of equivalents of the following claims.

1. A method for blinding a dosage form comprising printing one or more layers of ink to said dosage form such that the original markings on said dosage form are masked.

2. The method of claim 1 wherein the dosage form comprises a formulated medication or a placebo.

3. The method of claim 2 wherein the dosage form comprises a formulated medication or a placebo used to conduct clinical trials.

4. The method of claim 1 wherein the dosage form is selected from: a tablet; a soft gelatin capsule; and a hard gelatin capsule.

5. The method of claim 4 wherein the dosage form is a hard gelatin capsule.

6. The method of claim 1 wherein said printing is achieved by a process selected from: spin printing and linear printing.

7. The method of claim 6 wherein said printing is achieved by spin printing.

8. (canceled)

9. A method for blinding a dosage form via an overprinting process comprising the steps of:
a) loading said dosage form into a printing machine;
b) adjusting the print speed on said printing machine;
c) adjusting the print location on said printing machine;
d) printing; and
e) repeating said overprinting process as necessary to achieve complete masking.

10. The method of claim 9 wherein said printing machine is selected from: a spin printing machine and a linear printing machine.

11. The method of claim 10 wherein said printing machine is a spin printing machine.

12. The method of claim 9 wherein the dosage form comprises a formulated medication or a placebo.

13. The method of claim 12 wherein the dosage form comprises a formulated medication or a placebo used to conduct clinical trials.

14. The method of claim 9 wherein the dosage form is selected from: a tablet; a soft gelatin capsule; and a hard gelatin capsule.

15. The method of claim 14 wherein the dosage form is a hard gelatin capsule.

16. The method of claim 9 wherein said print speed ranges from about 2 to about 20 rpm.

17. (canceled)

18. (canceled)

19. A method for blinding a dosage form via an overprinting process comprising the steps of:
a) preparing said dosage form to be loaded into a printing machine;
b) staging said dosage form into said printing machine;
c) printing; and
d) repeating said printing as necessary to achieve complete masking.

20. The method of claim 19 wherein said dosage form comprises a formulated medication or a placebo used to conduct clinical trials.

21. The method of claim 20 wherein said placebo is filled with inert excipients.

22. The method of claim 20 wherein said formulated medication is a comparator which is removed from a market container.

23. The method of claim 19 wherein said staging occurs in said printing machine hopper.
